(19) United States
(12) Patent Application Publication
(10) Pub. No.: US 2010/0172997 A1
(43) Pub. Date: Jul. 8, 2010

(54) GOLD, SILVER, AND COPPER NANOPARTICLES STABILIZED IN BIOCOMPATIBLE AQUEOUS MEDIA

(75) Inventors: Mohammad A. Omary, Denton, TX (US); Zhibing Hu, Denton, TX (US); Sreekar Marpu, Denton, TX (US); Oussama Elbjeirami, Dahran (SA)

Correspondence Address: CHALKER FLORES, LLP, 2711 LBJ FRWY, Suite 1036, DALLAS, TX, 75234, US

(73) Assignee: UNIVERSITY OF NORTH TEXAS, Denton, TX (US)

(21) Appl. No.: 12/650,254

(22) Filed: Dec. 30, 2009

Related U.S. Application Data
(60) Provisional application No. 61/141,526, filed on Dec. 30, 2008.

(51) Int. Cl. A61K 9/14; A61B 18/18; A61M 37/00; A61P 43/00

(52) U.S. Cl. 424/489; 604/20; 977/777

(57) ABSTRACT

The present invention includes metal nanoparticles composition and methods of making and using the same by converting a metal (I) to a metal (0) and forming one or more metal nanoparticles from the metal (0). The one or more metal nanoparticles are stabilized with one or more biocompatible stabilizers to prevent agglomeration and make them amenable for biomedical applications.

### CROSS-REFERENCE TO RELATED APPLICATIONS

This application claims benefit of U.S. Provisional application Ser. No. 61/141,526 filed on Dec. 30, 2008, which is incorporated herein by reference in its entirety.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

### Technical Field of the Invention

The present invention relates in general to the field of metal nanoparticle synthesis and in particular, methods of synthesizing nanoparticles using environmentally benign non-toxic materials for stabilization in aqueous media toward use in pharmaceutical and biological applications.

Without limiting the scope of the invention, its background is described in connection with metal nanoparticle synthesis. The performance and function of metal nanostructures depends size, shape, composition, and structure and have many uses in numerous fields; however, the synthesis of metal nanoparticles poses potential environmental and biological risks. The syntheses methods found in the literature generally involve the reduction of metal ions using reducing agents such as hydrazine, sodium borohydride (NaBH₄), and dimethyl formamide, which are highly reactive and toxic chemicals. Generally, the literature method for the synthesis of near-infrared (NIR)-absorbing gold nanoparticles is a 3-step process (Chem. Mater. 2003, 15, 1957): First, tetrachloroauric acid (HAuCl₄), cetyltrimethylammonium bromide (CTAB) and NaBH₄ are mixed to form a seed solution. Second, HAuCl₄, CTAB. benzyldimethylhexadecylammonium chloride (BDAC), ascorbic acid, and silver nitrate (AgNO₃) are used to form a growth solution. Third, the seed and growth solutions are mixed in fixed proportions. CTAB (stabilizer), NaBH₄ (reducing agent), AgNO₃ and CDAB (growth enhancers) are very toxic to both human cells and the environment. None of these toxic chemicals is used in the synthetic methods of the present invention.

### SUMMARY OF THE INVENTION

The instant invention provides a method of synthesizing nanoparticles using environmentally benign non-toxic materials for stabilization in aqueous media toward use in pharmaceutical and biological applications. The preparation of metal nanoparticles in this invention involves only environmentally benign, biocompatible and/or non-toxic materials.

In contrast to the methods used in the prior art, the syntheses method of the present invention of NIR-absorbing gold nanoparticles is a facile single-step method and involves significantly fewer chemicals compared to methods in the literature. Chemicals in literature methods including CTAB (stabilizer), NaBH₄ (reducing agent), AgNO₃ and CDAB (growth enhancers) are very toxic to both human cells and the environment. Environmental concerns and cell toxicity are of major concern in the chemicals used in the literature synthesis methods, which are not used in this invention. Minimizing the use of chemicals and effective replacement of these chemical ligands with biologically adaptable biomolecules will enhance all biological applications of gold nanoparticles. The present invention allows the syntheses of NIR-absorbing gold nanoparticles with about 700-1200 nm plasmon absorptions in a single-step from a single starting precursor.

In one embodiment the present invention includes compositions and methods of making metal nanoparticles comprising the steps of: converting a metal (I) to a metal (0); forming one or more metal nanoparticles from the metal (0); and stabilizing the one or more metal nanoparticles with one or more polymer stabilizers to prevent agglomeration. In one aspect, the metal(I) precursor is a gold (I) complex, silver (I) complex or salt, copper (I) complex or salt, or combinations thereof. In another aspect, the metal(I) comprises Au(THT)Cl (where THT=tetrahydrothiophene), AuMe₂SCl, or Au(CO)Cl. In another aspect, the step of converting comprises photoreduction reaction, thermolysis reaction or both to convert the metal (I) to the metal (0). In another aspect, the one or more stabilizers comprise one or more polymers, one or more gels, one or more surfactants, or a combination thereof. In another aspect, the one or more stabilizers comprise or are selected from agarose, hydrogels, PAA (poly acrylic acid), PVA (poly vinyl alcohol), Chitosan, PNIPAM (Poly-N-isopropyl acrylamide), PNIPAM-aa (poly-N-isopropyl acrylamide-acrylic acid), PNIPAM-allylamine (Poly-N-isopropylacrylamide-allylamine), PAMAM (Polyamidoamine), PEG (Poly ethyleneglycol), alginic acid, HPC (hydroxyl propylcellulose), or a combination thereof. In another aspect, the method further comprises the step of conjugating the one or more metal nanoparticles to an active agent to form a site specific active agent delivery complex.

Another embodiment of the present invention is a metal nanoparticle made by the process comprising the steps of: converting a metal (I) to a metal (0); forming one or more metal nanoparticles from the metal (0); and stabilizing the one or more metal nanoparticles with one or more polymeric stabilizers to prevent agglomeration, wherein the synthesis occurs in solvents, solutions and using materials that are biocompatible, non-toxic, or both. In one aspect, the method further comprises the step of conjugating the one or more metal nanoparticles to an active agent to form a site specific active agent delivery complex. In another aspect, the method further comprises the step of conjugating the one or more metal nanoparticles to a binding agent for use as a diagnosis complex. In one aspect, the one or more metal nanoparticles are used in surface enhanced Raman scattering for the detection of small molecules. In another aspect, the method further comprises the step of conjugating the one or more metal nanoparticles to a cell surface for cell imaging.

Yet another embodiment are nanoparticles and methods of tuning the plasmon absorption energies and intensities and corresponding variation of the size and shape of metal nanoparticles comprising the steps of: converting a metal (I) to a metal (0); forming one or more metal nanoparticles from the metal (0); adjusting one or more parameters selected from pH, ionic strength, reaction time, irradiation time, temperature, and combinations thereof to adjust the tuning the plasmon absorption energies and intensities and corresponding variation of the size and shape of the one or more metal nanoparticles to adjust a plasmon absorption energy, an intensity or a combination thereof; and stabilizing the one or more metal nanoparticles with one or more stabilizers to prevent agglomeration. In one aspect, the step of converting comprises photoreduction reaction, thermolysis reaction or both to convert the metal (I) to the metal (0). In another aspect, the one or more stabilizers comprise one or more polymers, one or more gels, one or more surfactants, or a combination thereof. In another aspect, the one or more stabilizers comprises agarose, hydrogels, PAA (poly acrylic acid), PVA(poly vinyl alcohol), Chitosan, PNIPAM (Poly-N-isopropyl acrylamide), PNIPAM-aa (poly-N-isopropyl acrylamide-acrylic acid), PNIPAM-allylamine (Poly-N-isopropylacrylamide-allylamine), PAMAM (Polyamidoamine), PEG (Poly ethyleneglycol), HPC (hydroxyl propylcellulose), or a combination thereof. In another aspect, the method further comprises the step of conjugating the one or more metal nanoparticles to an active agent to form a site specific active agent delivery complex. In another aspect, the metal(I) comprises Au(THT)Cl (where THT=tetrahydrothiophene), AuMe₂SCl, or Au(CO)Cl. In another aspect, the one or more stabilizers comprise modified microgels comprising one or more functional groups. In another aspect, the metal (I) comprises a metal selected from the group consisting of titanium, gold, platinum, palladium, nickel, silver, copper or manganese. In another aspect, the metal (0) comprises at least one metal atom selected from the group consisting of aluminum, antimony, arsenic, barium, beryllium, bismuth, cadmium, calcium, cerium, chromium, cobalt, copper, dysprosium, erbium, europium, gadolinium, gallium, gold, hafnium, holmium, indium, iridium, iron, lanthanum, lead, lithium, lutetium, magnesium, manganese, mercury, molybdenum, neodymium, nickel, niobium, osmium, palladium, platinum, potassium, praseodymium, rhenium, rhodium, rubidium, ruthenium, samarium, scandium, silver, strontium, tantalum, technetium, terbium, titanium, thallium, thorium, thulium, tin, tungsten, uranium, vanadium, ytterbium, yttrium, zinc, and zirconium.

In another embodiment the invention includes a method of making metal nanoparticles comprising the steps of: converting a metal (I) to a metal (0); forming one or more metal nanoparticles from the metal (0); and stabilizing the one or more metal nanoparticles with one or more stabilizers to prevent agglomeration, wherein the entire synthesis is performed using reagents and solutions that are biocompatible. In yet another embodiment, the present invention includes nanoparticles and methods of treating a tissue comprising: selecting a tissue in need of therapy; contacting the tissue with a therapeutically effective amount of metal nanoparticles made by: converting a metal (I) to a metal (0); forming one or more metal nanoparticles from the metal (0); and stabilizing the one or more metal nanoparticles with one or more stabilizers to prevent agglomeration, wherein the nanoparticles are produced with non-toxic materials that are biocompatible. In one aspect, the therapy is selected from photothermal therapy, and drug delivery.

The instant invention also provides a metal nanoparticle made by the process of converting a metal (I) to a metal (0) and forming one or more metal nanoparticles from the metal (0). The one or more metal nanoparticles are stabilizing with one or more stabilizers to prevent agglomeration. The present invention provides a method of tuning the plasmon absorption energies and intensities and corresponding variation of the size and shape of metal nanoparticles by converting a metal (I) to a metal (0) and forming one or more metal nanoparticles from the metal (0). One or more parameters selected from pH, ionic strength, reaction time, irradiation time, temperature, centrifugation, sonication, and combinations thereof are adjusted to adjust the tuning the plasmon absorption energies and intensities and corresponding variation of the size and shape of the one or more metal nanoparticles in order to adjust the plasmon absorption energy, intensity or a combination thereof. The one or more metal nanoparticles are stabilized with one or more stabilizers to prevent agglomeration. The present invention also provides a method for using metal nanoparticles produced from non-toxic materials for photothermal therapy, including cell killing and drug delivery.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic of the synthesis of gold nanoparticles stabilized within the representative biologically-benign polymer microgel PNIPAM. Part-1 describes conditions for syntheses of PNIPAM-co-allylamine/acrylic acid based hydrogels. Part-2 describes syntheses of gold nanoparticles with visible or NIR absorption in the above microgel/hydrogel starting with Au(THT)Cl or Au(Me₂S)Cl as precursor under different possible conditions.

FIGS. 2a and 2b are schematics of the synthesis of gold nanoparticles stabilized within different commercially-available benign biopolymers and at different reaction conditions.

FIG. 3 shows the structure of the benign biopolymer monomers whose structures are shown in FIG. 2.

FIGS. 4-15 are images of absorption spectra in the UV/Vis/NIR regions for different gold nanoparticle samples prepared under different conditions.

FIGS. 16-20b are TEM and SEM images of the gold nanoparticles.

FIG. 21 shows absorption spectra of selected NIR-absorbing toxin-free Au NPs made by thermolysis of Au(THT)Cl (left) and Au(Me₂S)Cl (right) in PNIPAM-NH₂ microgel. The calculation shown illustrates that the use of a broad-band NIR lamp (covering the entire absorption range indicated by the peak area) instead of a common diode laser (providing only monochromatic light at 800 nm indicated by the peak height) may provide greater intensity for photothermal therapy applications for such Au NPs.

FIGS. 22a and 22b show proof-of-concept demonstrations of the usefulness of the non-toxic NIR-absorbing gold nanoparticles in this invention for photothermal therapy and drug delivery applications.

FIG. 23 demonstrates the stability of Au NPs prepared by the methods of this invention under physiological pH and temperature conditions (M-199 medium/PBS buffer) contrasted with the instability of particles prepared by the common methods.

FIGS. 24a and 24b are graphs showing lack of toxicity of the Au NPs in this invention contrasted with extreme toxicity of commercial particles following conventional methods of syntheses.

FIG. 25 is an EDAX elemental analysis data for gold/silver (Au/Ag) hybrid nanoparticles made using the methods of this invention. Note the signals due to both Ag and Au. The Cu signals are due to the sample holder and should be ignored.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The invention relates to syntheses of gold colloidal nanoparticles in aqueous media. The gold colloidal nanoparticles produced by the instant invention provide unique optical, electronic and molecular recognition properties that make them suitable agents for various biological applications. The prior art gold colloidal nanoparticles synthesis procedures use the Au(III) species HAuCl₄ as the starting precursor for the syntheses of gold colloidal nanoparticles and requiring the aid of strong chemical reducing agents like NaBH₄ to reduces Au(III) to Au(0), which is then stabilized to prevent agglomeration by a variety of stabilizers that include polymers (e.g., PAA, Chitosan), gels (e.g., PNIPAM, PAMAM, or PEG), and surfactants (e.g., CTAB or BDAC). In contrast, the instant invention provides a method of synthesizing gold colloidal nanoparticles using Au(I) complexes such as Au(THT)Cl, AuMe₂SCl, and Au(CO)Cl as promising starting precursors for the syntheses of gold colloidal nanoparticles. The photoreduction and thermolysis reactions of the instant invention achieve this property due to the reduction of Au(I)→Au(0) in our precursors as compared to that from Au(III)→Au(0) in the prior art precursors. The instant invention provides tunable plasmon absorption capabilities across visible and NIR regions with emphasis on minimizing the use of potentially harmful chemicals like CTAB, NaBH₄, BDAC, and AgNO₃. This invention also teaches the syntheses of the gold colloidal nanoparticles without aid of chemical reducing agents and under conditions that include photolysis, thermolysis, and stirring at ambient conditions. The instant invention includes biologically benign polymers that include Chitosan, agarose, PAA, PVA, along with “smart” thermo-responsive/stimuli-sensitive polymer hydrogels such as PNIPAM-aa and PNIPAM-allylamine as stabilizing agents for gold nanoparticles derived from Au(I) complexes as precursors. Although the instant example references gold nanoparticles the skilled artisan will recognize that this applies to other metals that can be converted from a (I) state to a (0) state, e.g., silver.

As used herein, the term “aqueous” refers to a liquid mixture containing water, among other components.

As used herein, the term “bioactive agent” or “active agent” are used interchangeably and refer to a substance used in an application that is therapeutic in nature, such as methods for treating disease in a patient. Non-limiting examples of active agents include but are not limited to, anti-inflammatory agents, blood modifiers, anti-platelet agents, anti-coagulation agents, immune suppressive agents, anti-neoplastic agents, anti-cancer agents, anti-cell proliferation agents, and nitric oxide releasing agents, polynucleotides, polypeptides, oligonucleotides, gene therapy agents, nucleotide analogs, nucleoside analogs, polynucleic acid decoys, and therapeutic antibodies.

As used herein, the term “biocompatible” refers to the material, substance, compound, molecule, polymer, solutions, solvents, compositions, reagents or systems, which does not cause severe toxicity, severe adverse biological reaction, or lethality in an animal when administered at reasonable doses and rates. Typically, biocompatible materials are biologically inert and non-toxic in that they do not generate any immune and/or inflammatory reaction when provided to an organism such as an animal or human.

As used herein, the term “therapeutically-effective amount” refers to that amount of the nanoparticles of the present invention in an amount sufficient to modulate one or more of the symptoms of the condition or disease being treated (with or without additional therapeutic intervention, e.g., infrared energy directed at a target site or loading the nanoparticles with an active agent). A “therapeutically effective amount” and/or dosage range of the nanoparticles of the present invention used in the method of treatment of the invention may be determined by one of ordinary skill in the art via known criteria including target tissue, age, weight, and response of the individual patient, and interpreted within the context of the disease being treated and/or prevented.

The metal nanoparticles synthesized by the method of the instant invention may be used for site specific drug delivery devices where gold nanoparticles are bio-conjugated to drugs or other active agents and then release them at specific sites of interest by various mechanisms such as photothermal volume phase transitions.

The metal nanoparticles synthesized by the method of the instant invention may also be used for surface enhanced Raman scattering (SERS) as diagnostic tools for the detection of small molecules, distinguishing cancerous cells from non-cancerous cells as a result of the strong scattering of gold nanoparticles by their to specific antibodies that bind only to cancerous cells. In addition, the gold nanoparticles can be conjugates to oligonucleotides for use as a detectable signature for detection of precise DNA sequence. Furthermore, the metal nanoparticles may be used for immunolabeling; imaging of cells and biomolecules; and recognition of proteins based on the interactions between metal nanoparticles-antibody conjugates (e.g., specifically gold nanoparticles-antibody conjugates) and their corresponding antigens.

The present invention provides a method of making gold nanoparticles (Au NPs) from common starting materials of Au(I) complexes (e.g., Au(Me₂S)Cl, Au(THT)Cl, and Au(CO)Cl, where Me₂S is dimethyl sulfide and THT is tetrahydrothiophene) in aqueous media that include biocompatible polymers and hydrogels (e.g., PNIPAM=poly(N-isopropylacrylamide), Chitosan, agarose, and poly(acrylic acid)).

The present invention also provides stable gold nanoparticles that have non-agglomerating behavior on long standing under ambient conditions, as deduced from persistence of the physical color of the samples without precipitation, the absorption spectra, and TEM and SEM images.

In addition, the present invention provides Au(I) complexes to produce and tune the properties of gold nanoparticles stabilized in biologically-compatible media without adding chemical reducing agents or other toxic reagents. In contrast, all common preparation methods of gold nanoparticles known to the skilled artisan rely on chemical reduction of the Au(III) precursor tetrachloroauric acid (HAuCl₄) by adding a hazardous reducing agent such as NaBH₄; other harmful reagents such as CTAB=hexadecyltrimethlyammoniumbromide, BDAC=benzyldimethylammoniumchloride, and silver nitrate (AgNO₃) are used to further grow the particle size (e.g., long nanorods) to make the gold nanoparticles absorb near-infrared (NIR) light, as needed for some biological applications. The instant invention does not utilize any of these harmful reagents. The instant invention provides a method of tuning the plasmon absorption energies and intensities and corresponding variation of the size and shape of the so formed gold nanoparticles by altering the reaction conditions, stabilizing biopolymer, and/or the starting Au(I) complex precursor.

The instant invention provides a method of synthesizing gold nanoparticles starting from Au(I) precursor Au(Me₂S)Cl, which is available commercially. The synthesis of the stabilizing agent PNIPAM-co-allylamine (denoted henceforth as “PNIPAM-allylamine”) or PNIPAM-co-acrylic acid (denoted henceforth as “PNIPAM-aa”) microgels is based on a literature procedure (Hu, Z.; Gang, H.; Angew. Chem. Int. Ed. 2003, 42, 4799-4802). PNIPAM is known to the skilled artisan as representative biologically-benign polymer and include polymers of Chitosan, PAA, PEG, PVA, agarose, HPC, NIPA, which are available commercially. The syntheses of gold nanoparticles in microgels and different polymers involve the addition of 3.5-5 mg of the Au(I) precursor directly as a solid to the stirred solution of 0.2 weight percent microgel or 3-5 weight percent solution of different polymer solutions made by the addition of millipore water. The solution containing both the precursor and the stabilizing agent (e.g., PNIPAM microgel or another polymer) leads to the formation of gold nanoparticles under three conditions. The first condition is photolysis. Photolysis employs a UV photolysis lamp maintaining the temperature constant around 22° C. and using a cold water bath for about 20 minutes to initiate the formation of gold nanoparticles in solution; the change of color from colorless to violet/purple indicates formation of gold nanoparticles. The second condition is thermolysis where the same reaction can be performed by heating the complete reaction mixture to about 40° C. for 20 minutes. The third condition includes ambient conditions, where the reaction is also achievable simply by stirring the solution under ambient conditions of light and temperature for about 45 minutes. The completion of the reaction is indicated by the intense purple/violet color of the solution in about 45 minutes by photochemistry/thermochemistry and about 150 minutes under ambient conditions. Varying the starting Au(I) precursor changes the reaction times; for example, using Au(CO)Cl leads to instant formation of gold nanoparticles even under ambient conditions. The solutions are highly stable for long duration storage the solutions can be centrifuged at 1000-1200 rpm for 5 minutes to remove any unreacted starting materials. Though the presence of the ligand Me₂S (boiling point=38° C.) is debatable, it can be easily removed by heating the solution to the boiling point of the ligand, which leaves the gold nanoparticle solutions completely free of any unwanted/hazardous materials while preserving the physical and chemical properties of colloidal gold nanoparticles without change. In the case of Au(CO)Cl, the dissociated ligand (CO) is a gas molecule so it evaporates in the hood even without heating. Absorption measurements in the UV/Vis/NIR region give primary information about the size range of the particles; SEM/TEM microscopy are then used to provide more accurate/quantitative information about the formation of gold nanoparticles with tunable size and shape, which can be controlled by experimental parameters such as the identity and concentration of the starting precursor and/or stabilizing agent.

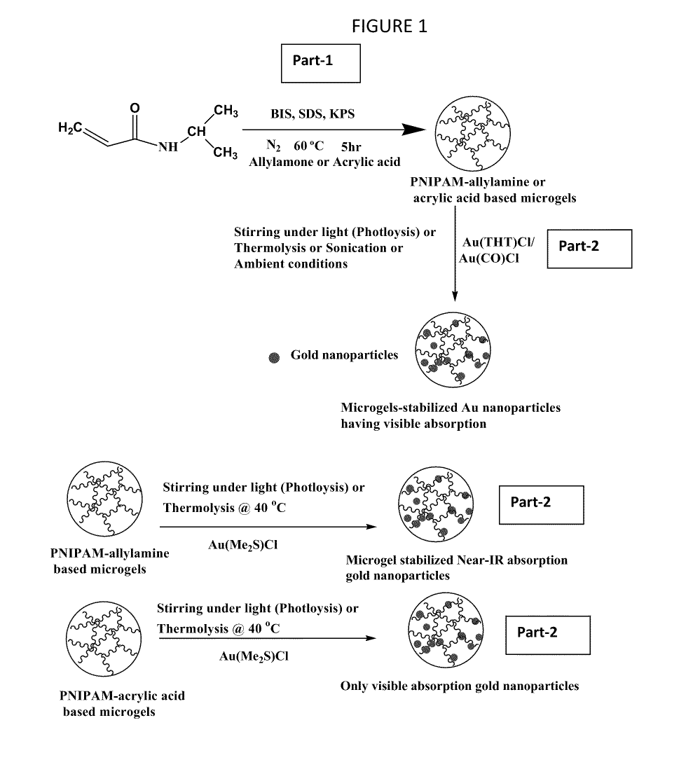

The present invention provides a method of synthesizing gold nanoparticles. FIG. 1 is a schematic of the synthesis of gold nanoparticles stabilized within the representative biologically-benign polymer microgel PNIPAM. A biologically-benign polymer PNIPAM microgel spherical in shape is formed (e.g., PNIPAM-allylamine or PNIPAM-acrylic acid microgel) from the specific monomers. Au(Me₂S)Cl, Au(CO)Cl, or Au(THT)Cl is added to the microgel during stirring and under light (photolysis), heat (thermolysis), or ambient conditions to form a PNIPAM microgel-stabilized gold nanoparticles.

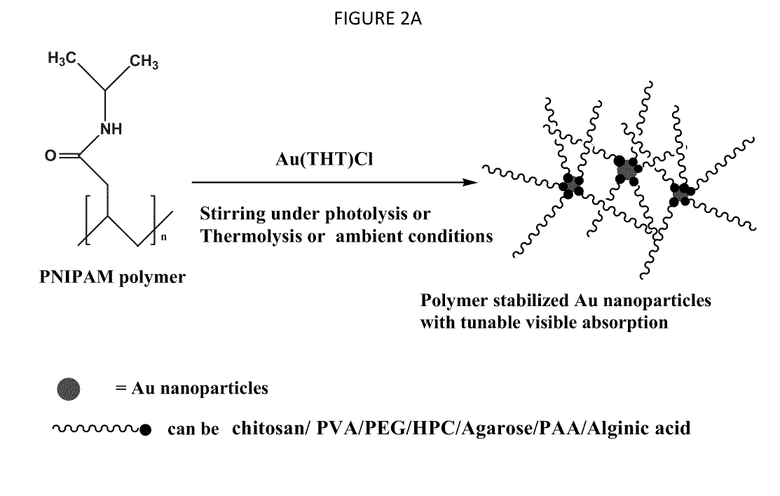

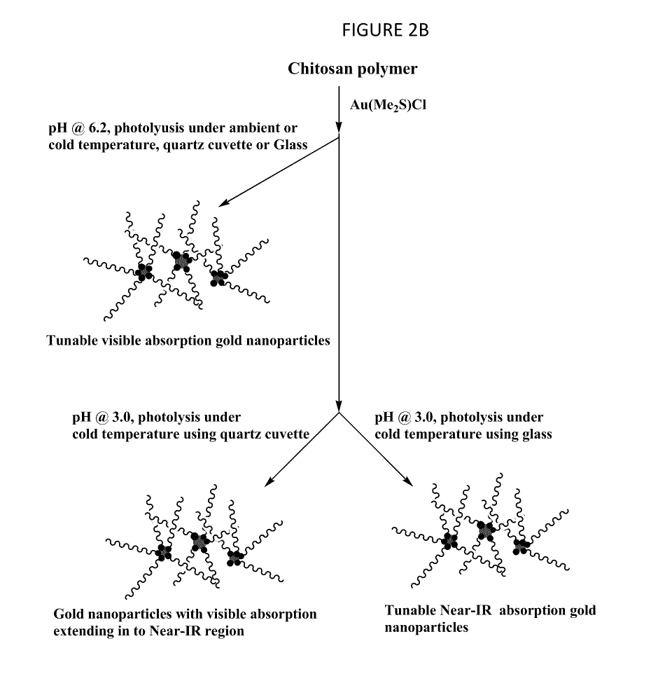

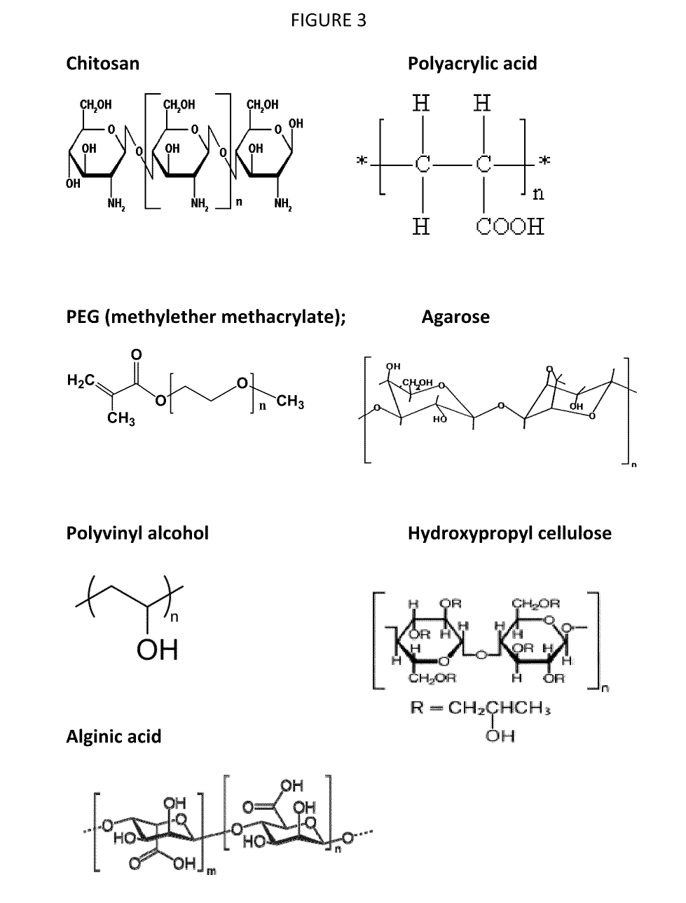

FIG. 2 is a schematic of the synthesis of gold nanoparticle stabilized within different commercially-available benign linear biopolymers under different experimental conditions. FIG. 2a shows the mechanism in such linear biopolymers whose structures are shown in FIG. 3 while FIG. 2b illustrates the variability of some experimental conditions during the synthesis. The relevant linear biopolymer may be mixed with Au(Me₂S)Cl, Au(CO)Cl, or Au(THT)Cl under conditions that include stirring under light (photolysis), heat (thermolysis), or ambient conditions to form polymer stabilized gold nanoparticles. The biopolymer can be seen surrounding the gold nanoparticles. The linear biopolymers may be chitosan, polyacrylic acid (PAA), PEG (methylether methacrylate), agarose, polyvinyl alcohol, hydroxypropyl cellulose, alginic acid, or other known polymer many of which are shown in FIG. 3. The variability of the polymers and synthetic conditions in FIGS. 1-3 are useful for the control of the Au NP properties and the versatility of their uses. The microgel spherical matrix of PNIPAM is more compact compared to linear polymer matrixes, giving rise to narrower absorption peaks suggesting more uniform particles compared to those formed in polymer-stabilized samples. On the other hand, each of the other biopolymers offers other advantages so as to make using it as a stabilizer of Au NPs worthwhile. For example, alginic acid is a natural biodegradable biopolymer available in varieties of alginates, which are extracted from sea weeds. Chitosan is an FDA-approved derivative produced by deacetylation of chitin, which is the structural element in the exoskeleton of crustaceans (crabs, shrimp, etc.) and cell walls of fungi. Poly(ethylene glycol) or PEG is produced by the interaction of ethylene oxide with water, ethylene glycol or ethylene glycol oligomers; it is used in a variety of products including laxatives, skin creams, cetomacrogol, and sexual lubricants, frequently combined with glycerin. Agarose (also known as agar or agar agar) is a gelatinous substance derived from seaweed; nutrient agar is used throughout the world to provide a solid surface containing medium for the growth of bacteria and fungi. PAA is capable of absorbing many times its weight in water, and hence is used in disposable diapers. Hydroxypropyl cellulose (HPC) is a derivative of cellulose with both water solubility and organic solubility; it is used as a topical ophthalmic protectant and lubricant. Polyvinyl alcohol has excellent film forming, emulsifying, and adhesive properties; it is also resistant to oil, grease and solvent, and is odorless and nontoxic.

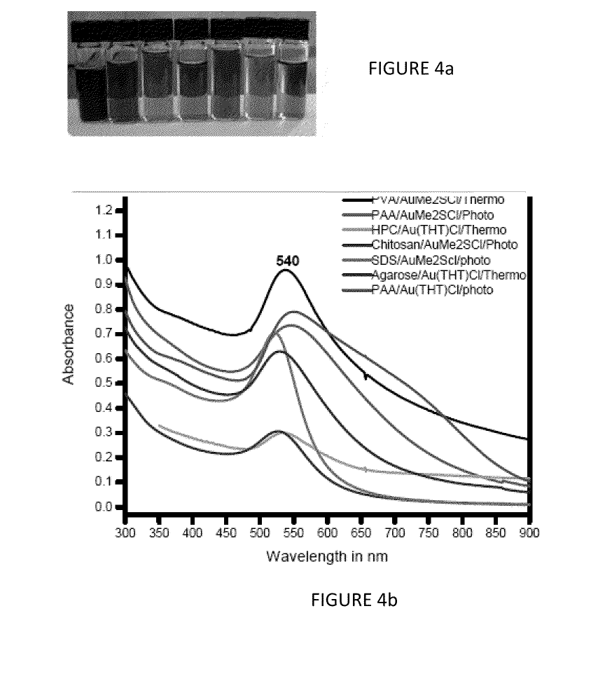

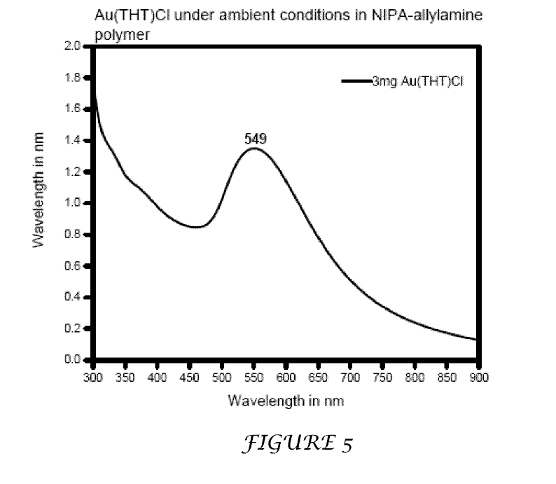

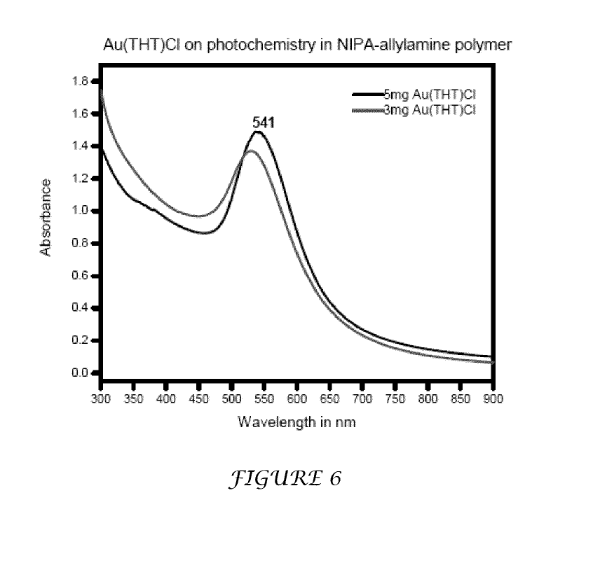

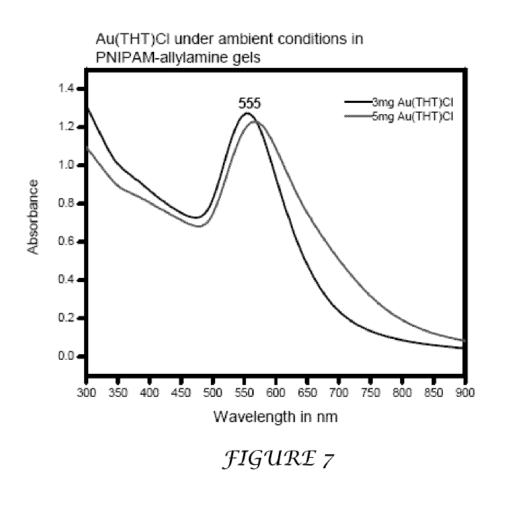

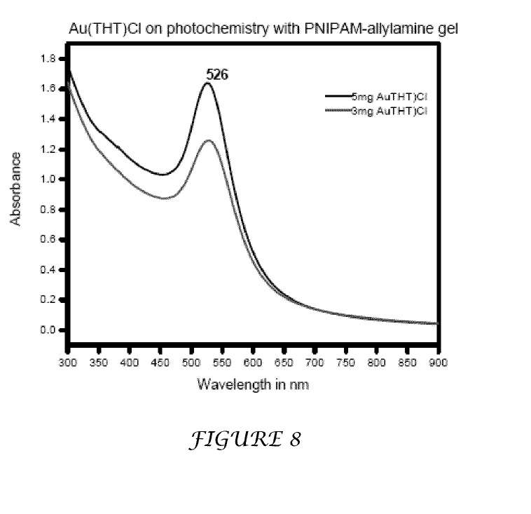

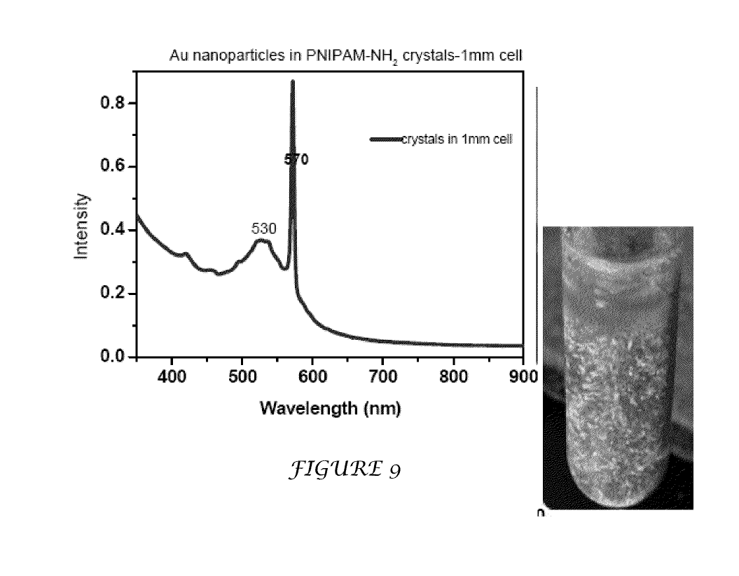

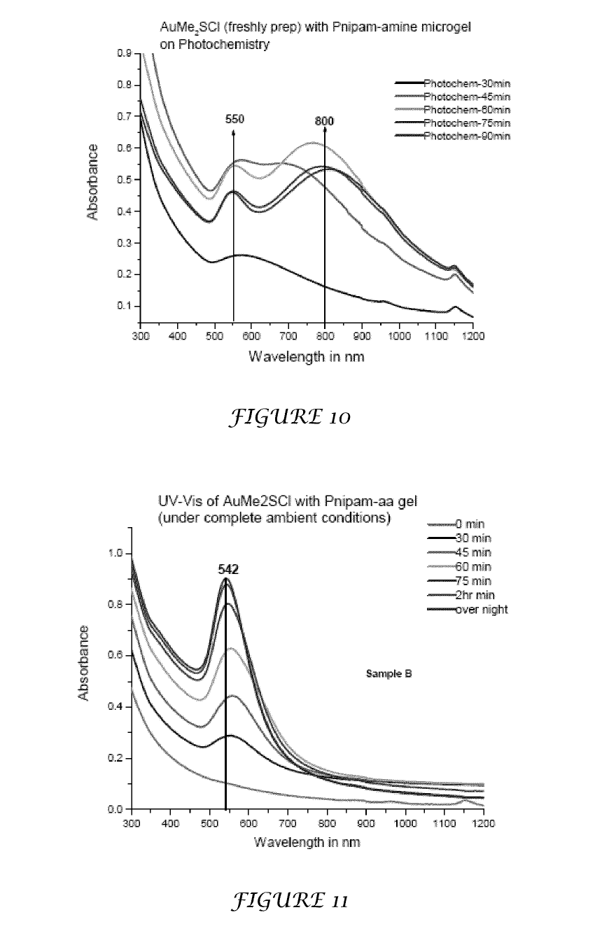

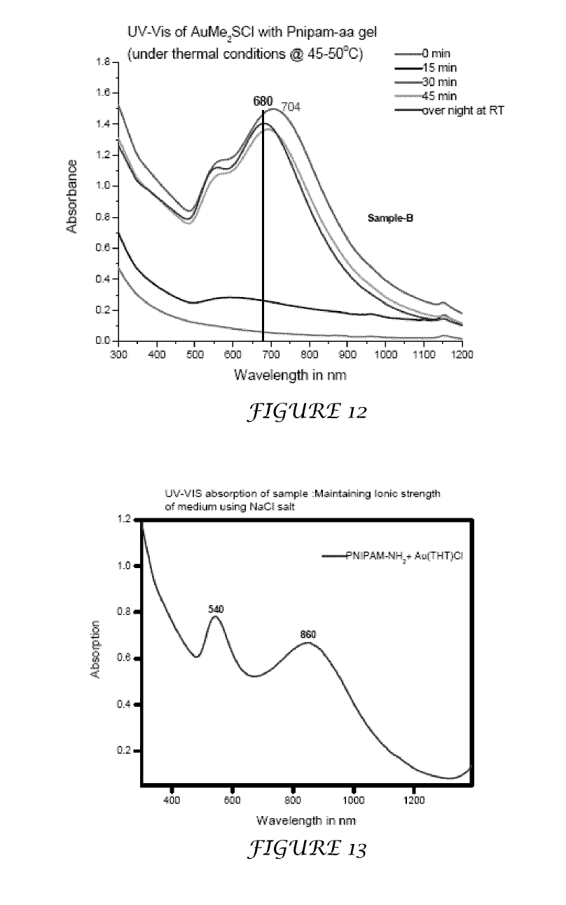

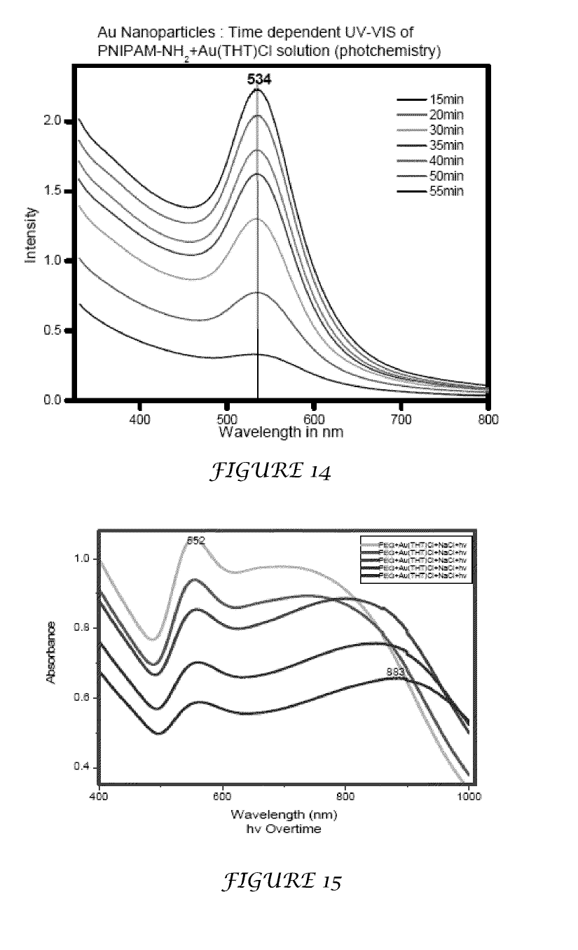

FIGS. 4-15 are images of absorption spectra in the UV/Vis/NIR regions for different gold nanoparticle samples prepared under different conditions. The production of gold nanoparticles is demonstrated through appearance of plasmon absorptions characterized by broad signals at wavelengths longer than 500 nm. The plasmonic absorptions in the visible region, typically between 500-600 nm, represent gold nanospheres. Variation in the plasmon absorption peaks gives rise to different visible colors for the solution containing the particles as seen in FIG. 4a which is an image of the sample vials. FIG. 4b is a graph of the absorption spectra in the UV/Vis/NIR regions for different gold nanoparticle samples prepared under different conditions showing a peak at about 540 nm s. FIG. 5 is a graph of the absorption spectra in the UV/Vis/NIR regions for Au(THT)Cl under ambient conditions in NIPA-allylamine Polymer. FIG. 6 is a graph of the absorption spectra in the UV/Vis/NIR regions for Au(THT)Cl on photochemistry in NIPA-allylamine polymer. FIG. 7 is a graph of the absorption spectra in the UV/Vis/NIR regions for Au(THT)Cl under ambient conditions in PNIPAM-allylamine gels. FIG. 8 is a graph of the absorption spectra in the UV/Vis/NIR regions for Au(THT)Cl on photochemistry with PNIPAM-allylamine gel. Some mixtures, where the hydrogel was crystalline, attained both the plasmon absorption of Au NPs and the Bragg diffraction of the gel simultaneously; see FIG. 9. FIG. 9 is a graph of the absorption spectra in the UV/Vis/NIR regions for gold nanoparticles in PNIPAM-NH₂ crystals-1 mm cell. FIG. 10 is a graph of the absorption spectra in the UV/Vis/NIR regions for AuMe₂SCl (freshly prep) with PNIPAM-amine microgel on Photochemistry. FIG. 11 is a graph of the absorption spectra in the UV/Vis/NIR regions for AuMe₂SCl with PNIPAM-aa gel (under complete ambient conditions. FIG. 12 is a graph of the absorption spectra in the UV/Vis/NIR regions for AuMe₂SCl with PNIPAM-aa gel (under thermal conditions @ 45-50° C.). FIG. 13 is a graph of the absorption spectra in the UV/Vis/NIR regions for a sample maintaining the ionic strength of medium using NaCl salt. FIG. 14 is a graph of the absorption spectra in the UV/Vis/NIR regions for gold nanoparticles with a time dependent UV-VIS of PNIPAM-NH₂+Au(THT)Cl solution (photochemistry). As seen in FIGS. 10, 12, 13 and 15, a significant situation arises when the absorption is controlled to extend to the near-infrared (NIR) at wavelengths of about 700 nm and longer; such absorptions (representing large gold nanoparticles) are particularly important for drug delivery and cancer treatment.

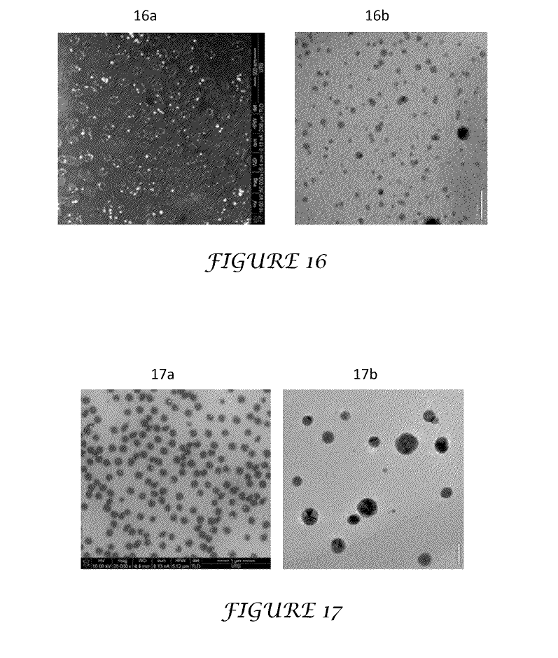

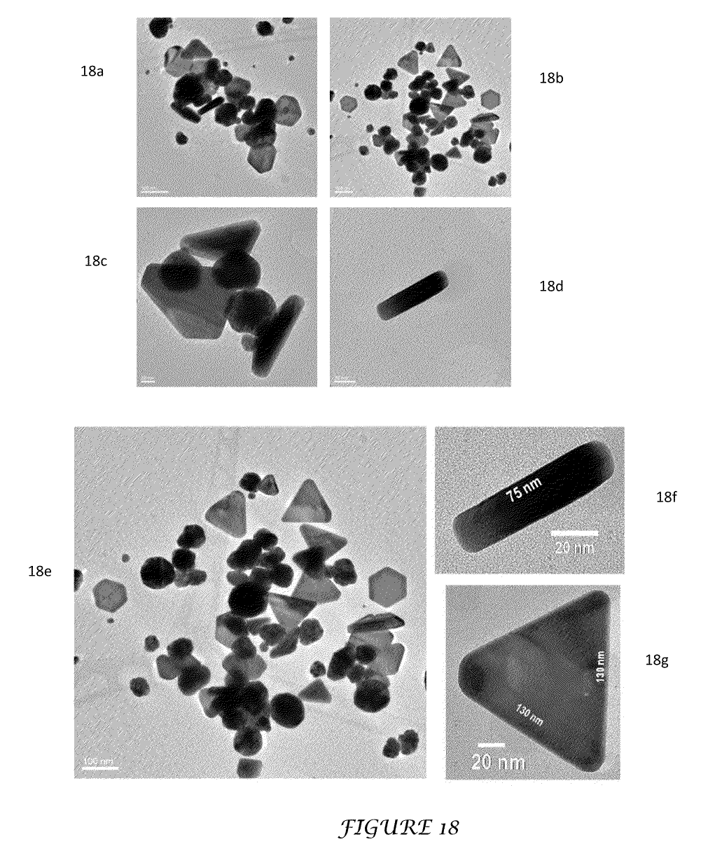

In addition, the size of the gold nanoparticles was also confirmed through TEM and SEM images; a few examples are shown in FIGS. 16-21. A variety of Au NPs are obtained, varying from small nanospheres with different radii (FIGS. 16-17) to large nanorods, nanoprism, as well as other large polyhedral and irregular shapes (FIGS. 18-20). These large gold nanoparticles are NIR-absorbing species, which are particularly important for drug delivery and cancer treatment.

FIG. 16a is an image of a field-emission SEM (FE-SEM) and FIG. 16b is a TEM image of gold nanoparticles prepared from Au(THT)Cl and PNIPAM-aa gel by stirring at under ambient conditions. The FE-SEM shows the spatial confinement of gold nanoparticles (bright spots) inside the gel (transparent shells).

FIG. 17a is an image of a field-emission SEM (FE-SEM) and FIG. 17b is a TEM image of gold nanoparticles prepared from Au(THT)Cl and PNIPAM-allylamine gel by photolysis.

FIGS. 18a-18g are TEM images of NIR-absorbing large gold nanoparticles, including rods, prisms, and polyhedral, prepared from Au(THT)Cl, PNIPAM-allylamine gel, and NaCl by photolysis.

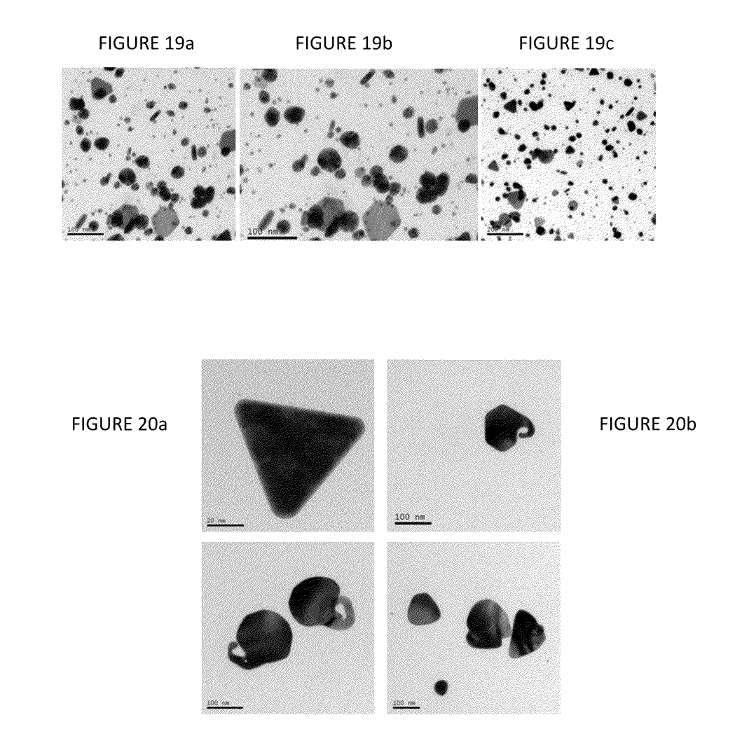

FIGS. 19a-19c are TEM images of NIR-absorbing large gold nanoparticles prepared from Au(Me₂S)Cl and PNIPAM-allylamine gel by photolysis.

FIGS. 20a-20d are TEM images of NIR-absorbing large gold nanoparticles, including prism-shaped particles and σ-shaped particles, prepared from Au(Me₂S)Cl and PNIPAM-allylamine gel by thermolysis.

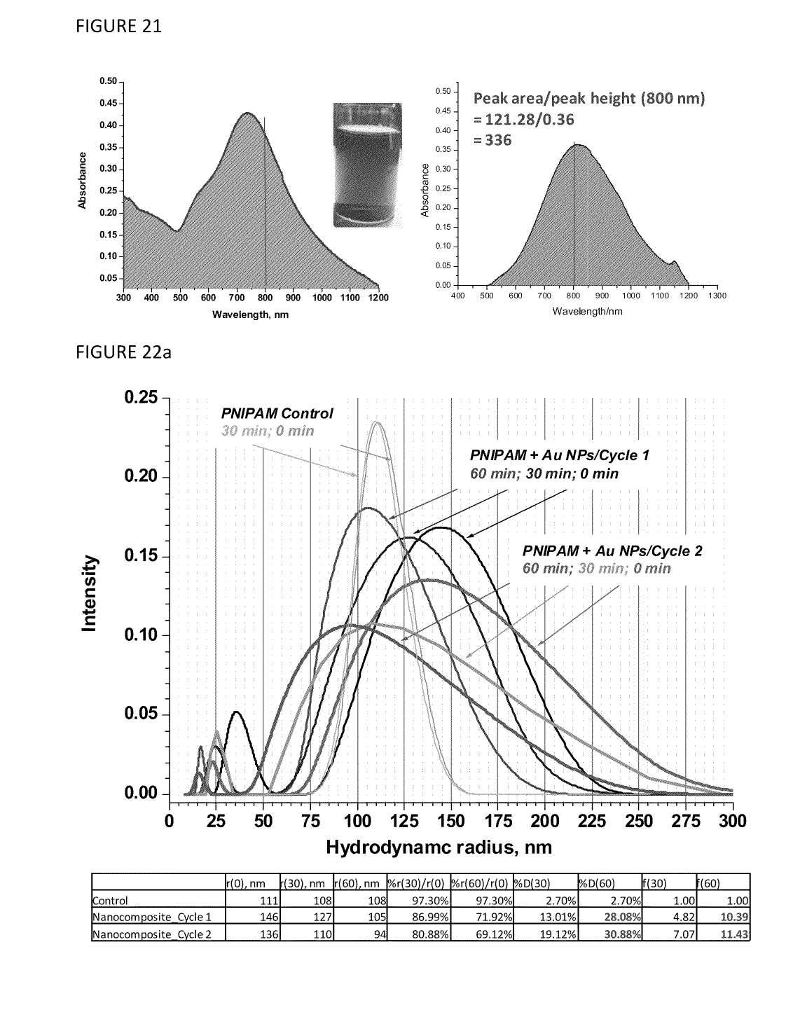

In addition the instant invention provides for the synthesize of gold nanorods with different aspect ratios, or to extend the absorption range further into the NIR so that the absorption will overlap with some particularly powerful NIR lasers (e.g., Nd/YAG, whose output is 1064 nm instead of less powerful diode lasers that emit at 800 nm) or broad-band emitting NIR lamps, whose advantage is illustrated in FIG. 21. These gold nanorods provide a mechanism to facilitate the drug delivery or cancer cell killing resulting from the heat generated by the NIR-absorbing gold nanoparticles embedded in the hydrogels upon their exposure to the laser.

The present invention may use a variety of Au(I) complex precursors. For example, Au(Me₂S)Cl may be used as a starting material with the Me₂S ligand removed after the formation of the gold nanoparticles by heating above its boiling point of 28° C. Au(CO)Cl may be used as a starting material with the CO ligand automatically removed after gold nanoparticles even under ambient conditions. Au(THT)Cl is the most common starting material for Au(I) complexes where the THT ligand is volatile and can be readily removed after gold nanoparticle formation by heating. Au(I) complexes as a general class can lead to formation of gold nanoparticles in biopolymers and hydrogels by following similar procedures illustrated in FIGS. 1-3.

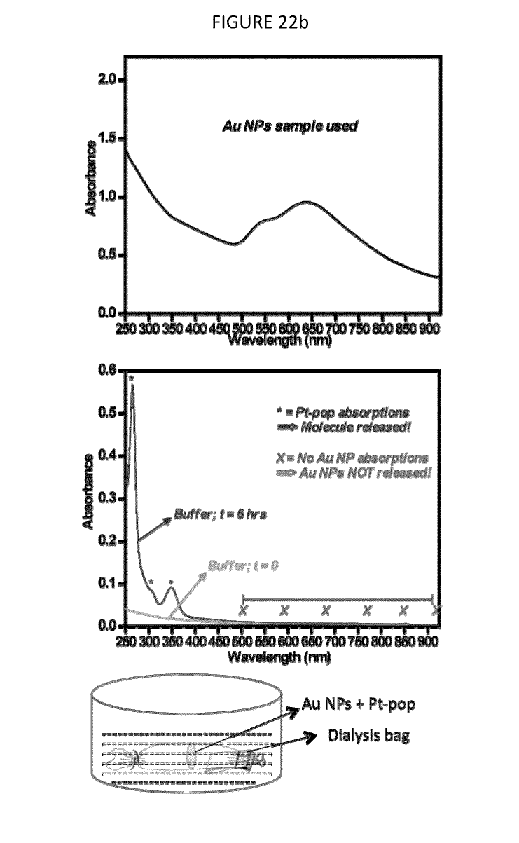

The metal nanoparticles of the instant invention may be used in photothermal therapy and drug delivery. FIG. 22 demonstrates this potential. FIG. 22a shows the change in the hydrodynamic radius of nanocomposite PNIPAM microgels impregnated with NIR-absorbing Au NPs upon irradiation with an NIR lamp. The data are shown for two cycles of the PNIPAM/Au NP nanocomposite sample as well as a control comprising PNIPAM alone without Au NPs. The sample shows rather significant de-swelling upon even modes irradiation with a low-power NIR lamp, whereas the control does not show any significant de-swelling. The de-swilling indicates a temperature increase beyond the volume phase transition temperature of PNIPAM and therefore demonstrates the potential of photothermal therapy (using the heat generated for example to kill cancer cells upon conjugation to the Au NPs) and drug delivery (the de-swelling can lead to release of a drug molecule co-entrapped in the nanocomposite). FIG. 22b provides further validation of the drug delivery application showing the release of the tetrakis (μ-diphosphito)-diplatinate (II) (Pt-pop) drug molecule upon irradiation or thermal heating of PNIPAM gels impregnated with both Au NPs and this drug molecule.

In addition, the metal nanoparticles of the instant invention may be used as multifunctional contrast agents with both targeting and delivery moieties. For example, literature studies utilized toxic Au NPs to distinguish the presence of visible filapodia in natural tissue (Nano Lett. 2007, 7, 1338-1343) so it will be more advantageous to perform such studies with the non-toxic Au NPs of this invention.

The metal nanoparticles of the instant invention that are small in size (absorb in the visible region) may be used for diagnosis of surface (e.g., skin) type cancer in place of small nanoparticles made by conventional synthetic methods Such spherical gold or silver nanoparticles conjugated to antibodies specifically targeted to cancer cells have been used to detect single malignant cells by dark field microscopy and spectrophotometry, whereas photothermal therapy of surface (skin) cancers could be accomplished by use of the small spherical gold or silver nanoparticles by exposure to low energy visible continuous wave (CW) lasers whereas deeper penetration beyond skin requires NIR-absorbing larger nanoparticles and thus NIR irradiation sources (e.g., Nano Lett. 2005, 5, 829-834; Cancer Lett. 2006, 239, 129-135; J. Am. Chem. Soc. 2006, 128, 2115-2120; Cancer Lett. 2008, 269, 57-66; Chem. Soc. Rev. 2008, 37, 1896-1908). The lack of cytotoxicity and greater versatility of the metal nanoparticles stabilized in multiple biocompatible media in the instant invention render them better replacement of the conventional more toxic nanoparticles for all these applications.

The present invention may use a variety of biopolymer and aqueous stabilizers. For example, the instant invention may use PNIPAM is a biocompatible polymer that can be derivatized with various functional groups; it represents the most extensively-studied stimulus-sensitive biopolymer nanoparticles as hydrogel materials in part due to phase change (swelling or contraction) in response to stimuli that change the temperature in either direction of its lower critical solution temperature (LCST). The instant invention may also use Chitosan which is an FDA-approved biopolymer; it is rather benign as it is derived from shrimp and other edible shellfish. Other biocompatible polymers and aqueous stabilizers that this technology pertains to include PAA, PEG, PVA, agarose, alginic acid, HPC, and SDS; these are representative examples of such materials that we surveyed and tested, not an exclusive list. In addition, a variety of functional groups on the PNIPAM microgels may be used. For example, varying the functional groups on PNIPAM gels from NH₂ to COOH or SH can improve the bio-conjugation ability of PNIPAM/Au NP hybrid nanocomposites.

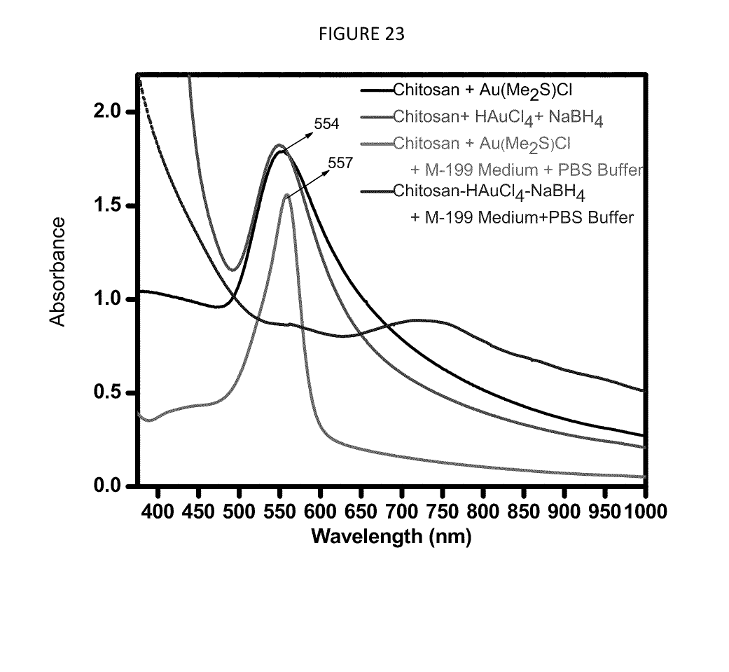

The present invention allows greater stability and biocompatibility of Au NPs by the methods described here compared to conventional synthetic methods. FIG. 23 illustrates this for chitosan-stabilized Au NPs synthesized by the method in this invention from Au(Me₂S)Cl vs. the conventional method from HAuCl₄ and NaBH₄. Upon adding the M-199 medium and PBS buffer to attain physiological conditions, the plasmon absorptions sharpened and the baseline decreased indicating sustained and actually enhanced stability of the Au NPs made following this invention. In contrast, the plasmon absorptions greatly broadened and the baseline increased for the Au NPs made following the conventional method, indicating their decreased stability and increased precipitation upon imposing physiological conditions.

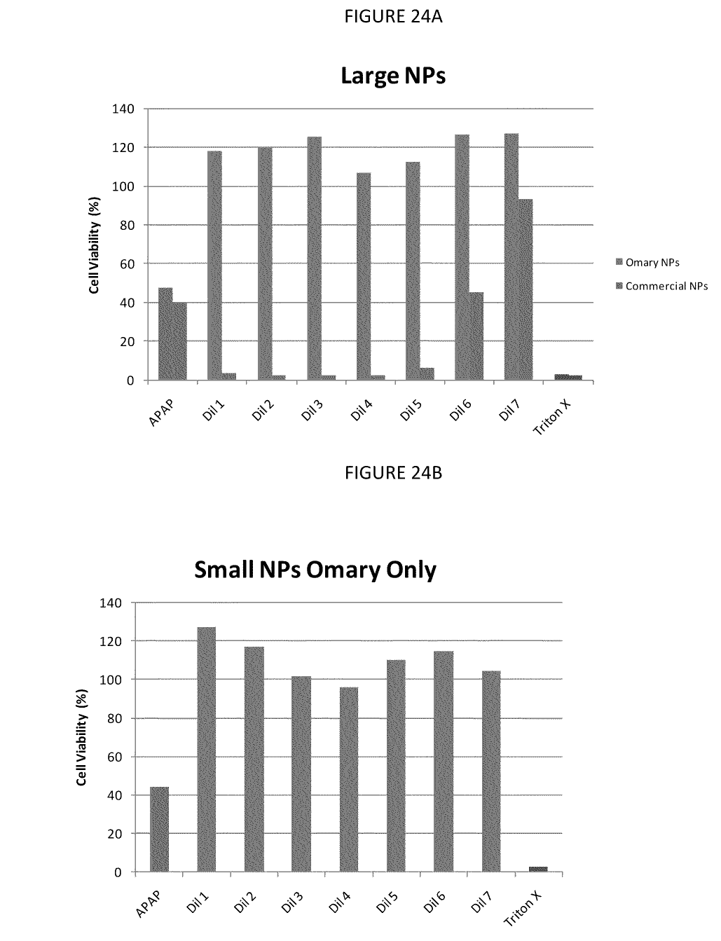

FIGS. 24a and 24b contrasts the cytotoxicity (NCL assay method) of the Au NPs synthesized by the method in this invention with the cytotoxicity of CTAB-stabilized commercial Au nanorods. The commercial CTAB-stabilized samples killed essentially all cells (<5% viability up to dilution 4), similar to the TritonX control cell killer and significantly worse than the APAP acetaminophen control that shows ˜40% viability. In contrast, the toxin-free Au NPs stabilized in FDA-approved chitosan made following this invention actually increased cell viability (promoted cell growth). Washing the commercial CTAB-stabilized Au NPs with biopolymers such PEG-NH₂ to remove excess CTAB did not lead to significant reduction of their cytotoxicity whereas no such workups are necessary for the non-toxic Au NPs made via this invention. The toxicity of the chemicals used in conventional methods of syntheses has been noted earlier in the literature as it has been found that the nanoparticles precursor HAuCl₄ and CTAB are toxic to cells at 10 nM concentrations (Small 2005, 1, 325-327).

Furthermore, the instant invention may vary the reaction conditions such as pH, ionic strength, reaction time, irradiation time, and/or temperature fine-tunes the properties of the gold nanoparticles. One particularly important subset of the embodiments pertains to those at physiological conditions of pH, ionic strength and temperature to make the hybrid nanocomposites suitable vehicles. In addition the instant invention provides a method of synthesis of gold nanoparticles in the absence of traces of reducing agents in biologically-compatible media and without by-products from the Au(I) precursors.

Size and shape variability to achieve strong plasmonic absorptions at wavelengths longer than about 700 nm to allow enhanced biological activities, e.g., photodynamic therapy, deep penetration of tissue, and heat-stimulated killing of tumors require such long absorption wavelengths for Au NP absorptions. Shorter-wavelength plasmonic absorptions of gold nanoparticles are still useful, particularly for skin cancer; see for example: El-Sayed et al., Cancer Letters 2006, 239, 129; J. Am. Chem. Soc. 2006, 128, 2115.

Chitosan-stabilized gold nanoparticles of the instant invention offer advantages for applications such as DNA delivery, heavy-metal sensing, medical diagnostics using films for surface-enhanced Raman spectroscopy (SERS), and other biological applications. All applications that utilize Chitosan-stabilized gold nanoparticles can be improved upon using our toxin-free Chitosan-stabilized gold nanoparticles instead of those known to the skilled artisan. For example, and each incorporated hereby reference: Hilborn, J. G.; Dutta, J.; Sugunan, A. “Heavy-Metal ion sensors using Chitosan-capped gold nanoparticles”, Science and Technology of Adv. Mat. 2005, 6, 335: Use of Chitosan serves dual purpose of providing sufficient steric hindrance ensuring stability of the colloid and also to functionalize the nanoparticles for use as sensors. Applications of gold nanoparticles as sensors are usually based on detecting the shifts in surface plasmon resonance (SPR) peak, due to either change in the dielectric constant around the nanoparticles as a result of adsorption of analyte molecules, or due to analyte-induced agglomeration of the nanoparticles. Kim, Y. H.; Yi, K. H.; Bahadur, K. C.; Bhattarai, R. S. “Hydrophobically modified Chitosan/gold nanoparticles for DNA delivery”, J. Nanopart. Res. 2008, 10, 151: Potentiality of Chitosan as a non-viral gene carrier has been extensively considered. In acidic pH the protonated amine groups of Chitosan can effectively bind to DNA and condense in to microparticles. Aroca, R. F.; Dos Santos, D. S.; Goulet, P. J. G.; Pieczonka, P. W.; Oliveira, N. O. “Gold nanoparticles embedded, self-sustained Chitosan films as substrates for surface enhanced raman scattering” Langmuir 2004, 20, 10273: Self sustained, biodegradable Chitosan films containing Au nanostructures fabricated for trace analysis using surface-enhanced Raman scattering. Yang, X.; Huang, H. “Chitosan mediated syntheses of gold nanoparticles multilayer”, Colloids and Surfaces. A: Physicochem. Eng. Aspects 2003, 226, 77: Syntheses of Au NPs using modified Chitosan.

The instant invention provides PNIPAM microgel-loaded gold nanoparticles that are particularly useful for drug delivery and other applications due to their photothermally-triggered volume phase transition. All applications that utilize PNIPAM-stabilized gold nanoparticles can be improved upon using the toxin-free PNIPAM-stabilized gold nanoparticles of the instant invention instead of those known to the skilled artisan. For example, and each incorporated hereby reference: Kumacheva, E.; Fava, D.; Sanson, N.; Das, M. “Microgels loaded with gold nanorods: Photothermally triggered volume phase transition under physiological conditions”, Langmuir, 2007, 23, 196. Lee, T. R.; Kim, J. H. “Hydrogel-Templated growth of large gold nanoparticles: Syntheses of thermally responsive hydrogel-Nanoparticle composites”, Langmuir, 2007, 23, 6504. Long, X.; Tian, C.; Peng, Y.; Zheng, Z.; Deng, Z.; Zhao, X. “A kind of smart gold nanoparticles-hydrogel composite with tunable thermo-switchable electrical properties”, New J. Chem. 2006, 30, 915. Willner, I.; Bourenko, T.; Shipway, N. A.; Gabai, R.; Yissar, V. P. “Gold nanoparticles/hydrogel composites with solvent-switchable electronic properties”, Adv. Mat. 2001, 13, 1320. Shi, L.; Zhang, W.; Zheng, P.; Jiang, X. “Thermoresponsive hydrogel of Poly (glycidyl methacrylate-co-N-isopropylacrylamide) as a Nanoreactor of gold nanoparticles”, J. Poly. Sci. A: Poly. Chem. 2007, 45, 2812. Lee, R. T; Kim, J. “Thermo- and pH-Responsive Hydrogel-coated Gold Nanoparticles” Chem. Mater. 2004, 16, 3647. Long, X.; Peng, Y.; Deng, Z.; Ding, X.; Zhao, X. “Thermoswitchable Electronic Properties Of a gold Nanoparticle/Hydrogel Composite”, Macromol. Rapid Commun. 2005, 26, 1784. Cho, K.; Kim, Y. D.; Cho, C. E. “Thermally responsive poly(N-isopropylacrylamide) monolayer on gold: syntheses, surface characterization, and protein interaction/adsorption studies”, Polymer 2004, 45, 3195.

PEG polymer-stabilized gold nanoparticles of the instant invention are particularly useful due to the special biocompatibility of the polymer stemming from its biological inertness and the documented use for such hybrid systems in the treatment of rheumatoid arthritis in addition to other pharmaceutical applications. All applications that utilize PEG-stabilized gold nanoparticles can be improved upon using the toxin-free PEG-stabilized gold nanoparticles of the instant invention instead of those described in the literature. For example, and each incorporated hereby reference: Kataoka, K.; Nagasaki, Y.; Otsuka, H. “PEGylated nanoparticles for biological and pharmaceutical applications”, Adv. Drug Deliv. Reviews 2003, 55, 403.

Agarose-stabilized gold nanoparticles of the instant invention are particularly useful because, being biologically benign, agarose ensures nondegradation of probe molecules and its gelation properties provide easy film formation for on-chip bio-sensing applications. All applications that utilize agarose-stabilized gold nanoparticles can be improved upon using the toxin-free agarose-stabilized gold nanoparticles of the instant invention instead of those described in the literature. For example, and each incorporated hereby reference: Guha, S.; Chandrasekhar, M.; Kattumuri, M. “Agarose-stabilized gold nanoparticles for surface enhanced Raman spectroscopic detection of DNA nucleosides”, Appl. Phys. Lett. 2006, 88, 153114. Ozaki, Y.; Ai, K.; Lu, L. “Environmentally friendly syntheses of highly monodisperse biocompatible gold nanoparticles with urchin-like shape”, Langmuir 2008, 1058. Au(Me₂S)Cl is available from Sigma-Aldrich. Au(CO)Cl is available from Strem. Au(THT)Cl is prepared using a previously described literature method (Usón, R.; Laguna, A. In Organometallic Syntheses; King, R. B., Eisch, J. J., Eds.; Elsevier: Amsterdam, 1986). In addition, the instant invention provides a method for using biologically benign polymers such as glucose, cellulose, starch, and polyacrylamide gels, thus offering a broader range of biologically benign stabilizers for the formation of gold nanoparticles.

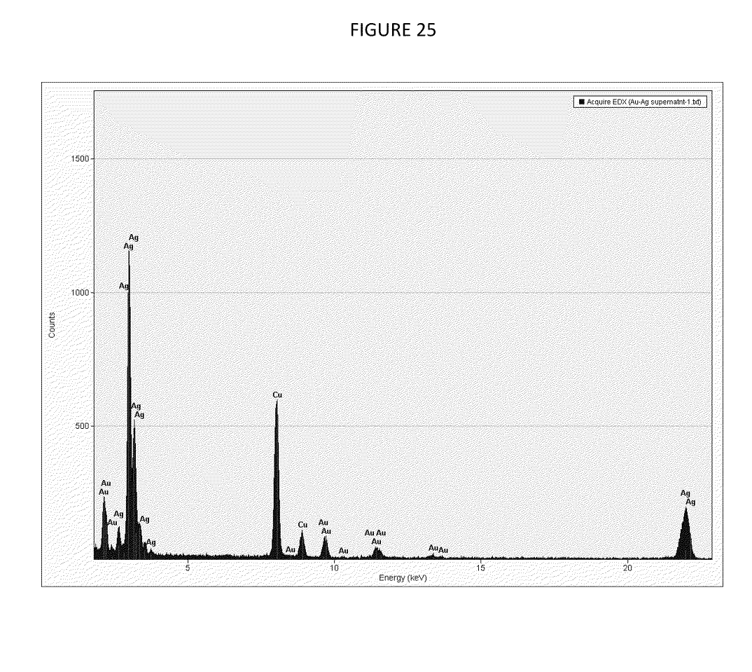

While the descriptions above focused mostly on the synthesis and biomedical applications of gold nanoparticles synthesized from Au(I) complexes as precursors for the sake of illustration, the same methods can be applied for the synthesis of silver and copper nanoparticles from Ag(I) and Cu(I) precursors, as well as the synthesis of hybrid gold/silver, gold/copper, silver/copper, and gold/silver/copper complexes. FIG. 25 illustrates EDAX elemental analysis data for Au/Ag hybrid nanoparticles synthesized and characterized via similar methodologies to those discussed above. The chemistry and d-electronic count (d¹⁰) of the three metals in their monovalent (+1) state make this generalization feasible.

The instant invention provides a method of making metal nanoparticles by converting a metal (I) precursor to a metal (0) and forming one or more metal nanoparticles from the metal (0) upon their controlled aggregation. The one or more metal nanoparticles are stabilized with one or more stabilizers to prevent agglomeration beyond the nanoscale. The metal(I) may be Au(THT)Cl, AuMe₂SCl, Au(CO)Cl or a plurality of Au(I) complexes with different ligands, as well as analogues thereof from Ag(I) and Cu(I) precursors. The step of converting may include photoreduction reaction, thermolysis reaction or both to convert the metal (I) to the metal (0). Stabilizers may include one or more polymers, one or more gels, one or more surfactants, agarose, hydrogels, PAA, PVA, Chitosan, PNIPAM, PNIPAM-aa, PNIPAM-allylamine, PAMAM, PEG, CTAB, BDAC or a combination thereof. In addition the present invention may be in contact with one or more metal nanoparticles to an active agent to form a site specific active agent delivery complex. The compositions of the instant invention may be used to conjugating the one or more metal nanoparticles to an active agent to form a site specific active agent delivery complex, to a binding agent for use as a diagnosis complex, used in surface enhanced Raman scattering for the detection of small molecules, or in conjugating the one or more metal nanoparticles to a cell surface for cell imaging. The metal (I) precursor may include a metal selected from the group consisting of gold (I), silver (I), and copper (I) complexes with different ligands and counter ions. The metal (0) includes at least one metal atom selected from the group consisting of gold, silver, and copper.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of making metal nanoparticles in an aqueous, biocompatible solution comprising the steps of:
converting a metal (I) to a metal (0);
forming one or more metal nanoparticles from the metal (0); and
stabilizing the one or more metal nanoparticles with one or more polymer stabilizers to prevent agglomeration.

2. The method of claim 1, wherein the metal(I) precursor is a gold (I) complex, silver (I) complex or salt, copper (I) complex or salt, or combinations thereof.

3. The method of claim 2, wherein the metal(I) precursor comprises Au(tetrahydrothiophene)Cl, AuMe₂SCl, or Au(CO)Cl.

4. The method of claim 1, wherein the step of converting comprises photoreduction reaction, thermolysis reaction or both to convert the metal (I) to the metal (0).

5. The method of claim 1, wherein the one or more stabilizers comprise one or more polymers, one or more gels, one or more surfactants, or a combination thereof.

6. The method of claim 1, wherein the one or more polymer stabilizers comprises agarose, hydrogels, PAA (poly acrylic acid), PVA (poly vinyl alcohol), Chitosan, PNIPAM (Poly-N-isopropyl acrylamide), substituted PNIPAM (including PNIPAM-aa (poly-N-isopropyl acrylamide-acrylic acid), PNIPAM-allylamine (Poly-N-isopropyl acrylamide-allylamine), and PNIPAM-SH), PAMAM (Polyamidoamine), PEG (Poly ethylene glycol), alginic acid, HPC (hydroxyl propyl cellulose), or a combination thereof.

7. The method of claim 1, further comprising the step of conjugating the one or more metal nanoparticles to an active agent to form a site specific active agent delivery complex.

8. A metal nanoparticle made by the process comprising the steps of:
converting a metal (I) to a metal (0) in an aqueous solution;
forming one or more metal nanoparticles from the metal (0); and
stabilizing the one or more metal nanoparticles with one or more stabilizers to prevent agglomeration.

9. The method of claim 8, further comprising the step of conjugating the one or more metal nanoparticles to an active agent to form a site specific active agent delivery complex.

10. The method of claim 8, further comprising the step of conjugating the one or more metal nanoparticles to a binding agent for use as a diagnosis complex.

11. The method of claim 8, wherein the one or more metal nanoparticles are used in surface enhanced Raman scattering for the detection of small molecules.

12. The method of claim 8, further comprising the step of conjugating the one or more metal nanoparticles to a cell surface for cell imaging.

13. A method of tuning the plasmon absorption energies and intensities and corresponding variation of the size and shape of metal nanoparticles comprising the steps of:
converting a metal (I) to a metal (0) in an aqueous solution;
forming one or more metal nanoparticles from the metal (0);
adjusting one or more parameters selected from pH, ionic strength, reaction time, irradiation time, temperature, centrifugation, sonication, reaction vessel material, optical filters, and combinations thereof, to adjust at least one of the tuning of the plasmon absorption energies or intensities and corresponding variation of at least one of size or shape of the one or more metal nanoparticles to adjust a plasmon absorption energy, an intensity or a combination thereof; and
stabilizing the one or more metal nanoparticles with one or more stabilizers to prevent agglomeration.

14. The method of claim 13, wherein the step of converting comprises photoreduction reaction, thermolysis reaction or both to convert the metal (I) to the metal (0).

15. The method of claim 13, wherein the one or more stabilizers comprise one or more polymers, one or more gels, one or more surfactants, or a combination thereof.

16. The method of claim 13, wherein the one or more stabilizers is a polymer selected from agarose, hydrogels, PAA (poly acrylic acid), PVA (poly vinyl alcohol), Chitosan, PNIPAM (Poly-N-isopropyl acrylamide), substituted PNIPAM (including PNIPAM-aa (poly-N-isopropyl acrylamide-acrylic acid), PNIPAM-allylamine (Poly-N-isopropyl acrylamide-allylamine), and PNIPAM-SH), PAMAM (Polyamidoamine), PEG (Poly ethylene glycol), alginic acid, HPC (hydroxyl propyl cellulose), or a combination thereof.

17. The method of claim 13, further comprising the step of conjugating the one or more metal nanoparticles to an active agent to form a site specific active agent delivery complex.

18. The method of claim 13, wherein the metal(I) precursor is a gold (I) complex, silver (I) complex or salt, copper (I) complex or salt, or combinations thereof.

19. The method of claim 13, wherein the metal(I) comprises Au(THT)Cl, AuMe₂SCl, or Au(CO)Cl.

20. The method of claim 13, wherein the one or more stabilizers comprises modified microgels comprising one or more functional groups.

21. The method of claim 13, wherein the metal (I) comprises a metal selected from the group consisting of titanium, gold, platinum, palladium, nickel, silver, copper or manganese.

22. The method of claim 13, wherein the metal (0) comprises at least one metal atom selected from the group consisting of aluminum, antimony, arsenic, barium, beryllium, bismuth, cadmium, calcium, cerium, chromium, cobalt, copper, dysprosium, erbium, europium, gadolinium, gallium, gold, hafnium, holmium, indium, iridium, iron, lanthanum, lead, lithium, lutetium, magnesium, manganese, mercury, molybdenum, neodymium, nickel, niobium, osmium, palladium, platinum, potassium, praseodymium, rhenium, rhodium, rubidium, ruthenium, samarium, scandium, silver, strontium, tantalum, technetium, terbium, titanium, thallium, thorium, thulium, tin, tungsten, uranium, vanadium, ytterbium, yttrium, zinc, and zirconium.

23. A method of making metal nanoparticles comprising the steps of:
converting a metal (I) to a metal (0) in an aqueous, non-toxic solution;
forming one or more metal nanoparticles from the metal (0); and
stabilizing the one or more metal nanoparticles with one or more stabilizers to prevent agglomeration, wherein the entire synthesis is performed using reagents and solutions that are biocompatible.

24. A method of treating a tissue comprising:
selecting a tissue in need of therapy;
contacting the tissue with therapeutically effective amount of a metal nanoparticles made by:
converting a metal (I) to a metal (0);
forming one or more metal nanoparticles from the metal (0); and
stabilizing the one or more metal nanoparticles with one or more stabilizers to prevent agglomeration, wherein the nanoparticles are produced with non-toxic materials that are biocompatible.

25. The method of claim 24, wherein the therapy is selected from photothermal therapy, and drug delivery.
